SEQUENCE LISTING (19 sequences)

SEQ ID NO: 1 - protein, 8 aa, synthetic construct
GYTFTSFT

SEQ ID NO: 2 - protein, 8 aa, synthetic construct
INPNSGNT

SEQ ID NO: 3 - protein, 9 aa, synthetic construct
TRWGAGFDY

SEQ ID NO: 4 - protein, 5 aa, synthetic construct
SSVSY

SEQ ID NO: 5 - protein, 9 aa, synthetic construct
LQYHTYPLT

SEQ ID NO: 6 - protein, 116 aa, synthetic construct
QVQLRQSGAELARPGASVKMSCKASGYTFTSFTIHWVIQRPGQGLEWIGYINPNSGNTNYNQKFKDKATLTADKSSSTAYIQLNSLTSEDSAVYFCTRWGAGFDYWGQGTTLTVSS

SEQ ID NO: 7 - protein, 106 aa, synthetic construct
QIVLTQSPAIMSASPGEKVTISCSASSSVSYVYWYQQKLGSSPKPWISRTSNLASGVPARFSGSGSGTSYSLTISSMEAEDAVTYYCLQYHTYPLTFGGGTKLEIK

SEQ ID NO: 8 - protein, 446 aa, synthetic construct
QVQLRQSGAELARPGASVKMSCKASGYTFTSFTIHWVIQRPGQGLEWIGYINPNSGNTNYNQKFKDKATLTADKSSSTAYIQLNSLTSEDSAVYFCTRWGAGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, synthetic construct
QIVLTQSPAIMSASPGEKVTISCSASSSVSYVYWYQQKLGSSPKPWISRTSNLASGVPARFSGSGSGTSYSLTISSMEAEDAVTYYCLQYHTYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 116 aa, synthetic construct
QVQLVQSGAEVKRPGASVKVSCKASGYTFTSFTIHWVRQAPGQGLEWMGYINPNSGNTNYNQKFKDRVTLTADKSTSTAYMELNSLTSEDTAVYFCARWGAGFDYWGQGTLVTVSS

SEQ ID NO: 11 - protein, 106 aa, synthetic construct
EIVLTQSPAILSVSPGEKVTISCSASSSVSYVYWYQQKPGSAPKLLISRTSNLASGVPARFSGSGSGTDFSLTISSLQAEDAATYYCLQYHTYPLTFGGGTKLEIK

SEQ ID NO: 12 - protein, 446 aa, synthetic construct
QVQLVQSGAEVKRPGASVKVSCKASGYTFTSFTIHWVRQAPGQGLEWMGYINPNSGNTNYNQKFKDRVTLTADKSTSTAYMELNSLTSEDTAVYFCARWGAGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, synthetic construct
EIVLTQSPAILSVSPGEKVTISCSASSSVSYVYWYQQKPGSAPKLLISRTSNLASGVPARFSGSGSGTDFSLTISSLQAEDAATYYCLQYHTYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 450 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 219 aa, synthetic construct
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 446 aa, synthetic construct
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 220 aa, synthetic construct
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 725 aa, synthetic construct
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSGGGGSMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSGGGGSMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 19 - protein, 277 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

(19) United States
(12) Patent Application Publication
(10) Pub. No.: US 2026/0347493 A1
(43) Pub. Date: Nov. 5, 2026

(54) BLOCKING CD40 ANTIBODY AND USE THEREOF

(71) Applicant: HEFEI TG IMMUNOPHARMA CO., LTD., Hefei (CN)

(72) Inventors: Guoshuai Cao, Hefei (CN); Ying Cheng, Hefei (CN); Yangyang Li, Hefei (CN); Yuwei Wu, Hefei (CN)

(21) Appl. No.: 19/703,374

(22) Filed: Jun. 10, 2026

Related U.S. Application Data
(63) Continuation of application No. PCT/CN2024/131726, filed on Nov. 13, 2024.

(30) Foreign Application Priority Data
Dec. 13, 2023 (CN) 202311718869.5

(51) Int. Cl. C07K 16/28; A61K 47/68; A61P 37/02; C12N 15/85; G01N 33/68

(52) U.S. Cl. CPC C07K 16/2878; A61K 47/6849; A61P 37/02; C12N 15/85; G01N 33/6854; C07K 2317/565; C07K 2317/567; C07K 2317/76; G01N 2333/70578

(57) ABSTRACT

Provided are an anti-CD40 antibody and the use thereof. The antibody comprises heavy chain variable region CDR1, CDR2 and CDR3 sequences respectively shown as SEQ ID NO: 1, 2 and 3 or amino acid sequences having at least 80% identity with SEQ ID NO: 1, 2 and 3; and light chain variable region CDR1, CDR2 and CDR3 sequences respectively shown as SEQ ID NO: 4, 5 and 6 or amino acid sequences having at least 80% identity with SEQ ID NO: 4, 5 and 6.

### CROSS-REFERENCE TO RELATED APPLICATIONS

This application is a continuation of International Patent Application No. PCT/CN2024/131726 filed on Nov. 13, 2024, which claims priority to and benefits of Chinese patent application No. 202311718869.5, filed with China National Intellectual Property Administration on Dec. 13, 2023, the entire contents of which are incorporated herein by reference.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence_Listing.xml”, was created on Jun. 3, 2026, edited on Jun. 9, 2026, and is 21,436 bytes as measured in Microsoft Windows operating system.

### FIELD

The present disclosure relates to the field of antibodies. Particularly, the present disclosure relates to an antibody or antigen-binding fragment thereof capable of binding to cluster of differentiation 40 (CD40), and use thereof.

### BACKGROUND

CD40 is a type I membrane protein that is primarily expressed on the surface of antigen-presenting cells (APCs), including B cells, monocytes, macrophages, and dendritic cells. Studies have shown that CD40 is also expressed on the surface of platelets and certain non-hematopoietic cells, such as fibroblasts, endothelial cells, and smooth muscle cells, and is expressed on some types of tumor cells as well. Therapies targeting CD40 have been applied in immune-related diseases, including autoimmune diseases and cancer. CD40 ligand (CD40L) is a ligand of CD40 and is a type II membrane protein expressed on the surface of various cells, including activated CD4 T cells, CD8 T cells, NK cells, granulocytes, endothelial cells, smooth muscle cells, macrophages, and platelets.

The interaction between CD40 and CD40L induces humoral and cell-mediated immune responses. Upon binding of CD40L to CD40, activation signals are transmitted into APCs, thereby promoting APC activation, upregulating the expression of costimulatory ligands such as CD80 and CD86, and enhancing APC proliferation, which ultimately promotes immune activation. In autoimmune diseases, antagonistic anti-CD40 antibodies can be used to bind CD40 and suppress CD40-mediated activation signals, thereby alleviating disease progression in patients suffering from autoimmune disorders such as multiple sclerosis.

Therefore, there remains an urgent need in the field for therapeutic agents capable of interfering with CD40-CD40L binding and blocking CD40 signal transduction.

### SUMMARY

The present disclosure aims to solve, at least to some extent, at least one of the technical problems existing in the related art. Currently, antagonistic anti-CD40 antibodies such as Iscalimab (Novartis) and Ravagalimab (AbbVie) are undergoing clinical trials. Through research, the inventors found that Iscalimab and Ravagalimab induce APC activation and upregulate the expression of CD80, CD86, and the like, which is contrary to the intended purpose of these two antibodies.

In view of this, the inventors screened and identified a murine anti-CD40 monoclonal antibody exhibiting high binding activity to a human CD40 protein. In addition, the inventors humanized the constant region of the above monoclonal antibody while retaining the complementarity-determining regions (CDRs) of the murine anti-CD40 monoclonal antibody, to obtain a chimeric antibody. Further, the framework regions in the light chain variable region or heavy chain variable region of the chimeric antibody were humanized to obtain a fully humanized anti-CD40 antibody. The CD40 antibody specifically targets and binds to human CD40 protein, potently and effectively blocks the interaction between CD40 and CD40L, inhibits downstream CD40 signaling, and suppresses proliferation of B cells, without inducing the activation of APCs (such as B cells). Thus, the antibody has potential utility in immune-related diseases, including autoimmune diseases, transplant rejection, and cancer.

Accordingly, in a first aspect, the present disclosure provides an antibody or antigen-binding fragment thereof. According to embodiments of the present disclosure, the antibody or antigen-binding fragment thereof includes: a heavy chain variable region CDR1, a heavy chain variable region CDR2, and a heavy chain variable region CDR3 having amino acid sequences as set forth in SEQ ID NO: 1, SEQ ID NO: 2, and SEQ ID NO: 3, respectively, or amino acid sequences having at least 80% identity to SEQ ID NO: 1, SEQ ID NO: 2, and SEQ ID NO: 3, respectively; and a light chain variable region CDR1, a light chain variable region CDR2, and a light chain variable region CDR3 having amino acid sequences as set forth in SEQ ID NO: 4, SEQ ID NO: 5, and SEQ ID NO: 6, respectively, or amino acid sequences having at least 80% identity to SEQ ID NO: 4, SEQ ID NO: 5, and SEQ ID NO: 6, respectively. The antibody or antigen-binding fragment thereof according to embodiments of the present disclosure is capable of binding to human CD40 protein, blocking the interaction between CD40 and CD40L, inhibiting downstream CD40 signaling, and suppressing proliferation of B cells, without inducing activation of APCs (such as B cells), thereby effectively treating or preventing CD40-mediated diseases and facilitating relevant scientific research.

According to embodiments of the present disclosure, the above antibody or antigen-binding fragment thereof may further include at least one of the following additional technical features.

According to an embodiment of the present disclosure, the antibody or antigen-binding fragment thereof includes: a heavy chain variable region CDR1 having a sequence as set forth in SEQ ID NO: 1, a heavy chain variable region CDR2 having a sequence as set forth in SEQ ID NO: 2, a heavy chain variable region CDR3 having a sequence as set forth in SEQ ID NO: 3, a light chain variable region CDR1 having a sequence as set forth in SEQ ID NO: 4, a light chain variable region CDR2 having a sequence as set forth in SEQ ID NO: 5, and a light chain variable region CDR3 having a sequence as set forth in SEQ ID NO: 6.

According to an embodiment of the present disclosure, the antibody or antigen-binding fragment thereof includes: a heavy chain variable region as set forth in SEQ ID NO: 7 or SEQ ID NO: 11; and/or a light chain variable region as set forth in SEQ ID NO: 8 or SEQ ID NO: 12.

According to an embodiment of the present disclosure, the antibody or antigen-binding fragment thereof includes: 1) a heavy chain variable region as set forth in SEQ ID NO: 7 and a light chain variable region as set forth in SEQ ID NO: 8; or 2) a heavy chain variable region as set forth in SEQ ID NO: 11 and a light chain variable region as set forth in SEQ ID NO: 12.

According to an embodiment of the present disclosure, the antibody or antigen-binding fragment thereof includes a heavy chain framework region and a light chain framework region. At least a portion of at least one of the heavy chain framework region and the light chain framework region is from at least one of a murine antibody, a human antibody, a primate antibody, or a mutant thereof.

According to an embodiment of the present disclosure, the antibody or antigen-binding fragment thereof includes at least one of a heavy chain constant region or a light chain constant region. At least a portion of at least one of the heavy chain constant region and the light chain constant region is from at least one of a human antibody, a primate antibody, a murine antibody, or a mutant thereof.

According to an embodiment of the present disclosure, the light chain constant region and the heavy chain constant region are both from a murine IgG antibody or a mutant thereof, or a human IgG antibody or a mutant thereof.

According to an embodiment of the present disclosure, the light chain constant region and the heavy chain constant region are both from a murine IgG1 antibody, a murine IgG2a antibody, a murine IgG4 antibody, or a mutant thereof, or both from a human IgG1 antibody, a human IgG2 antibody, a human IgG3 antibody, a human IgG4 antibody, or a mutant thereof.

According to an embodiment of the present disclosure, the antibody or antigen-binding fragment thereof includes: a heavy chain having an amino acid sequence as set forth in any one of SEQ ID NO: 9 and SEQ ID NO: 13; and a light chain having an amino acid sequence as set forth in any one of SEQ ID NO: 10 and SEQ ID NO: 14.

According to an embodiment of the present disclosure, the antibody or antigen-binding fragment thereof includes: a heavy chain having an amino acid sequence as set forth in SEQ ID NO: 9; and a light chain having an amino acid sequence as set forth in SEQ ID NO: 10.

According to an embodiment of the present disclosure, the antibody or antigen-binding fragment thereof includes: a heavy chain having an amino acid sequence as set forth in SEQ ID NO: 13; and a light chain having an amino acid sequence as set forth in SEQ ID NO: 14.

According to an embodiment of the present disclosure, the antibody or antigen-binding fragment thereof includes at least one of a monoclonal antibody, a polyclonal antibody, a multimeric antibody, or a CDR-grafted antibody.

As used herein, the term “monoclonal antibody” refers to an antibody capable of recognizing only a single specific antigenic epitope. A typical monoclonal antibody includes two low-molecular-weight light chains and two high-molecular-weight heavy chains. The heavy chains (H chains) and the light chains (L chains) are linked via disulfide bonds to form a tetrapeptide-chain molecule. The amino-terminal (N-terminal) amino acid sequences of the heavy chain and light chain vary considerably and are referred to as variable regions (V regions), whereas the carboxyl-terminal (C-terminal) amino acid sequences are relatively conserved and are referred to as constant regions (C regions). The V regions of the light chain and heavy chain are referred to as VL and VH, respectively. In addition, the monoclonal antibody includes, but is not limited to, at least one of a single-chain antibody, a Fab antibody, an Fv antibody, a single-chain antibody, a single-domain antibody, or a minimal recognition unit.

As used herein, the term “CDR-grafted antibody” refers to an antibody in which the CDRs of a monoclonal antibody from one species are grafted into the variable region of an antibody from another species. For example, the CDRs of a murine monoclonal antibody may be grafted into the variable region of a human antibody to replace the CDRs of the human antibody, thereby conferring the antigen-binding specificity of the murine monoclonal antibody on the human antibody while reducing heterologous immunogenicity. It should be noted that the polyclonal antibodies and monoclonal antibodies described herein may be CDR-grafted antibodies.

According to an embodiment of the present disclosure, the monoclonal antibody includes at least one of a full-length antibody, an Fv antibody, a single-chain antibody, a Fab antibody, a single-domain antibody, or a minimal recognition unit.

According to an embodiment of the present disclosure, the antibody or antigen-binding fragment thereof is capable of binding an amino acid sequence as set forth in SEQ ID NO: 20.

In a second aspect, the present disclosure provides a nucleic acid molecule. The nucleic acid molecule encodes the antibody or antigen-binding fragment thereof according to the first aspect of the present disclosure. According to some specific embodiments of the present disclosure, the antibody or antigen-binding fragment thereof encoded by the nucleic acid molecule is capable of binding to human CD40 protein and thus effectively treating or preventing a CD40-mediated disease.

According to an embodiment of the present disclosure, the nucleic acid molecule is DNA.

It should be noted that those skilled in the art should understand that any nucleic acid mentioned in the specification and the claims of the present disclosure encompasses either one or both strands of complementary double-stranded molecule. For convenience, although in most cases only one strand is provided in the specification and the claims, the complementary strand is also actually disclosed. In addition, the nucleic acid sequence in the present disclosure includes both DNA and RNA forms, and thus disclosure of one form implies disclosure of the other form as well.

In a third aspect, the present disclosure provides an expression vector. The expression vector carries the nucleic acid molecule according to the second aspect of the present disclosure. The expression vector may include an optional control sequence operably linked to the nucleic acid molecule. The control sequence refers to one or more control sequences capable of directing expression of the nucleic acid molecule in a host. The expression vector according to embodiment of the present disclosure enables efficient, large-scale expression of the antibody or antigen-binding fragment thereof in suitable host cells.

As used herein, the term “operably linked” means that an exogenous gene is linked to a vector such that the control elements in the vector, such as a transcriptional control sequence and a translational control sequence, can exert their intended functions in regulating transcription and translation of the exogenous gene. When the nucleic acid molecule is linked to the vector, the nucleic acid molecule may be directly or indirectly linked to the control elements of the vector, provided that such control elements are capable of controlling translation, expression, or the like of the nucleic acid molecule. Of course, such control elements may directly originate from the vector itself or may be exogenous, i.e., not originate from the vector itself. Those skilled in the art should understand that the nucleic acid molecule encoding the antibody or antigen-binding fragment may be independently inserted into different vectors, and is commonly inserted into the same vector. For example, common vectors may be plasmids, bacteriophages, etc. According to some specific embodiments of the present disclosure, after the expression vector is introduced into an appropriate recipient cell, expression of the aforementioned antibody or antigen-binding fragment thereof can be effectively achieved under the control of a regulatory system, thereby enabling large-scale in vitro production of the antibody or antigen-binding fragment thereof.

In a fourth aspect, the present disclosure provides a method for preparing the antibody or antigen-binding fragment thereof according to the first aspect of the present disclosure. The method includes: introducing the expression vector according to the third aspect of the present disclosure into a cell; and culturing the cell under a condition suitable for protein expression and protein secretion to obtain the antibody or antigen-binding fragment. According to some specific embodiments of the present disclosure, the method enables efficient large-scale in vitro production of the antibody or antigen-binding fragment thereof.

According to some specific embodiments of the present disclosure, the method may further include at least one of the following additional technical features.

According to some specific embodiments of the present disclosure, the cell is not particularly limited, and may be either a prokaryotic cell or a eukaryotic cell.

According to some specific embodiments of the present disclosure, the cell is a eukaryotic cell.

According to some specific embodiments of the present disclosure, the eukaryotic cell is a mammalian cell. According to some specific embodiments of the present disclosure, when the cell is a eukaryotic cell, such as a mammalian cell, expression efficiency of a recombinant antibody is relatively high.

In a fifth aspect, the present disclosure provides a recombinant cell. The recombinant cell carries the nucleic acid according to the second aspect of the present disclosure or the expression vector according to the third aspect of the present disclosure, or is capable of expressing the antibody or antigen-binding fragment thereof according to the first aspect of the present disclosure. The recombinant cell is obtained by transfection or transformation with the expression vector. According to some specific embodiments of the present disclosure, under suitable conditions, the recombinant cell can efficiently express the antibody or antigen-binding fragment thereof in large quantities.

It should be noted that the recombinant cell of the present disclosure is not particularly limited and may be a prokaryotic cell, a eukaryotic cell, or a bacteriophage. The prokaryotic cell may be Escherichia coli, Bacillus subtilis, Streptomyces, Proteus mirabilis, or the like. The eukaryotic cell includes fungi such as Pichia pastoris, Saccharomyces cerevisiae, Schizosaccharomyces pombe, and Trichoderma, insect cells such as Spodoptera frugiperda cells, plant cells such as tobacco cells, and mammalian cells such as BHK cells, CHO cells, COS cells, and myeloma cells. In some embodiments, the recombinant cell of the present disclosure is preferably a mammalian cell, including BHK cells, CHO cells, NSO cells, or COS cells, but excluding animal germ cells, fertilized eggs, and embryonic stem cells.

It should be noted that, as used herein, the term “suitable conditions” refers to conditions suitable for expression of the antibody or antigen-binding fragment thereof of the present disclosure. Those skilled in the art will readily appreciate that the conditions suitable for expression of the antibody or antigen-binding fragment thereof include, but are not limited to, an appropriate transformation or transfection method, an appropriate transformation or transfection condition, healthy host cell state, suitable host cell density, suitable cell culture environments, and a suitable cell culture duration. Such suitable conditions are not particularly limited and may be optimized by those skilled in the art according to specific laboratory conditions.

According to an embodiment of the present disclosure, the recombinant cell is obtained by introducing the aforementioned expression vector into a host cell.

According to an embodiment of the present disclosure, the recombinant cell is a eukaryotic cell.

According to an embodiment of the present disclosure, the recombinant cell is a mammalian cell.

In a sixth aspect, the present disclosure provides an immunoconjugate. The immunoconjugate includes the antibody or antigen-binding fragment thereof according to the first aspect of the present disclosure and a therapeutic agent. As described above, the antibody or antigen-binding fragment thereof according to embodiments of the present disclosure is capable of effectively binding to CD40 protein and thus effectively treating or preventing a CD40-related disease. Accordingly, the immunoconjugate including the antibody or antigen-binding fragment thereof is likewise capable of binding human CD40 protein. The immunoconjugate exhibits favorable efficacy in preventing and/or treating a CD40-mediated disease.

According to an embodiment of the present disclosure, the immunoconjugate may further include at least one of the following additional technical features.

According to an embodiment of the present disclosure, the therapeutic agent is capable of preventing and/or treating a CD40-mediated disease or other diseases.

According to an embodiment of the present disclosure, the CD40-mediated disease is selected from transplant rejection, an autoimmune disease, an infectious disease, and cancer.

In a seventh aspect, the present disclosure provides a composition. The composition includes the antibody or antigen-binding fragment thereof according to the first aspect, the nucleic acid molecule according to the second aspect, the expression vector according to the third aspect, the recombinant cell according to the fifth aspect of the present disclosure, or the immunoconjugate according to the sixth aspect of the present disclosure. As described above, the antibody or antigen-binding fragment thereof according to some specific embodiments of the present disclosure is capable of effectively binding to human CD40 protein and effectively inhibiting CD40-mediated B-cell activation. Accordingly, the composition including the above substance is likewise capable of effectively binding to human CD40 protein and exhibits favorable efficacy in preventing and/or treating a CD40-mediated disease. The type of the composition is not particularly limited and may be a food composition or a pharmaceutical composition.

The compositions of the present disclosure may also be administered in combination with each other or with one or more additional therapeutic compounds, for example, in combination with a chemotherapeutic agent. Therefore, the composition may further include a chemotherapeutic agent. The antibody or antigen-binding fragment, or the immunoconjugate, of the present disclosure may also be combined with a second therapeutic agent. Examples of the second therapeutic agent include, but are not limited to, additional agents that inhibit CD40 activity (including other antibodies or antigen-binding fragments, peptide inhibitors, small-molecule antagonists, etc.) and/or agents that interfere with upstream or downstream signal transduction of CD40.

It should be noted that the composition encompasses combinations separated in time and/or space, provided that they are capable of acting together to achieve the objectives of the present disclosure. For example, the components contained in the composition may be administered to a subject as a whole or separately. When the components contained in the composition are administered separately to the subject, the individual components may be administered to the subject simultaneously or sequentially.

In an eighth aspect, the present disclosure provides a medicament. The medicament includes the antibody or antigen-binding fragment thereof according to the first aspect of the present disclosure, the nucleic acid molecule according to the second aspect of the present disclosure, the expression vector according to the third aspect of the present disclosure, the recombinant cell according to the fifth aspect of the present disclosure, the immunoconjugate according to the sixth aspect of the present disclosure, or the composition described in the seventh aspect. As described above, the antibody or antigen-binding fragment thereof according to some specific embodiments of the present disclosure is capable of effectively binding to human CD40 protein. Therefore, the medicament including an effective amount of the antibody or antigen-binding fragment thereof as an active ingredient or a series of substances thereof is likewise capable of effectively binding to human CD40 protein and exhibits favorable efficacy in preventing and/or treating a CD40-mediated disease.

According to embodiments of the present disclosure, the medicament may further include at least one of the following additional technical features.

According to an embodiment of the present disclosure, the medicament may further include a pharmaceutically acceptable carrier.

As used herein, the term “effective amount” or “effective dose” refers to an amount capable of producing a desired function or activity in a human and/or animal and that is acceptable to the human and/or animal.

The effective amount of the antibody or antigen-binding fragment thereof of the present disclosure may vary depending on factors such as the mode of administration and the severity of the disease to be treated. A preferred effective amount may be determined by those of ordinary skill in the art based on various factors (e.g., through clinical studies). Such factors include, but are not limited to, pharmacokinetic parameters of the active ingredient, such as bioavailability, metabolism, and half-life; the severity of the disease to be treated; the patient's body weight; the patient's immune status; and the route of administration. For example, depending on exigencies of the therapeutic situation, multiple divided doses may be administered daily, or the dosage may be proportionally adjusted.

As used herein, a “pharmaceutically acceptable” component is a substance that is suitable for use in humans and/or mammals without undue adverse side effects (e.g., toxicity, irritation, and allergic reactions), i.e., having a reasonable benefit/risk ratio. The term “pharmaceutically acceptable carrier” refers to a carrier used for administration of a therapeutic agent and includes various excipients and diluents.

The medicament of the present disclosure includes a safe and effective amount of the active ingredient of the present disclosure and a pharmaceutically acceptable carrier. Such carriers include, but are not limited to, saline, buffer, glucose, water, glycerol, ethanol, and a combination thereof. Generally, a pharmaceutical preparation should be compatible with the mode of administration, which may be oral administration, intranasal administration, intradermal administration, subcutaneous administration, intramuscular administration, intravenous administration, or intraperitoneal administration. The dosage form of the medicament of the present disclosure may be an injection, an oral formulation (tablets, capsules, and oral liquids), a transdermal agent, and a sustained-release agent. For example, the medicament is prepared by conventional methods using physiological saline or an aqueous solution containing glucose and other adjuvants. The medicament is preferably manufactured under sterile conditions. The antibody or antigen-binding fragment may be administered through intravenous infusion, intravenous injection, intramuscular injection, or subcutaneous injection.

In a ninth aspect, the present disclosure provides use of the antibody or antigen-binding fragment thereof according to the first aspect of the present disclosure, the nucleic acid molecule according to the second aspect of the present disclosure, the expression vector according to the third aspect of the present disclosure, the recombinant cell according to the fifth aspect of the present disclosure, the immunoconjugate according to the sixth aspect of the present disclosure, or the composition according to the seventh aspect of the present disclosure in the manufacture of a medicament for preventing and/or treating a CD40-mediated disease. As described above, the antibody or antigen-binding fragment thereof according to some specific embodiments of the present disclosure is capable of effectively binding to human CD40 protein. Therefore, the medicament including an effective amount of the antibody or antigen-binding fragment thereof or a series of substances thereof is likewise capable of effectively binding to human CD40 protein and exhibits favorable efficacy in preventing and/or treating a CD40-mediated disease.

In a tenth aspect, the present disclosure provides use of the antibody or antigen-binding fragment thereof according to the first aspect of the present disclosure, the nucleic acid molecule according to the second aspect of the present disclosure, the expression vector according to the third aspect of the present disclosure, the recombinant cell according to the fifth aspect of the present disclosure, the immunoconjugate according to the sixth aspect of the present disclosure, the composition according to the seventh aspect of the present disclosure, or the medicament according to the eighth aspect of the present disclosure in the prevention and/or treatment of a CD40-mediated disease. As described above, the antibody or antigen-binding fragment thereof according to some specific embodiments of the present disclosure is capable of effectively binding to human CD40 protein. Therefore, an effective amount of the antibody or antigen-binding fragment thereof or a series of substances thereof is likewise capable of effectively binding to human CD40 protein and exhibits favorable efficacy in preventing and/or treating a CD40-mediated disease

According to an embodiment of the present disclosure, the above use may also include at least one of the following additional technical features.

According to an embodiment of the present disclosure, the CD40-mediated disease includes transplant rejection, an autoimmune disease, an infectious disease, and cancer.

According to an embodiment of the present disclosure, the autoimmune disease includes, but is not limited to, at least one of systemic lupus erythematosus, rheumatoid arthritis, systemic vasculitis, scleroderma, dermatomyositis, autoimmune hemolytic anemia, autoimmune thyroid disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, pemphigoid, primary biliary cirrhosis, multiple sclerosis, or acute idiopathic polyneuritis.

According to an embodiment of the present disclosure, the cancer includes, but is not limited to, at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head and neck cancer.

In an eleventh aspect, the present disclosure provides a kit. According to an embodiment of the present disclosure, the kit includes the antibody or antigen-binding fragment thereof according to the first aspect of the present disclosure, the nucleic acid molecule according to the second aspect of the present disclosure, the expression vector according to the third aspect of the present disclosure, or the recombinant cell according to the fifth aspect of the present disclosure. As described above, the antibody or antigen-binding fragment thereof according to some specific embodiments of the present disclosure is capable of effectively binding to human CD40 protein. Therefore, the kit including the antibody or antigen-binding fragment thereof is capable of effective qualitative and/or quantitative detection of human CD40 protein. The kit according to the present disclosure may be used, for example, in assays involving the specific binding interaction between human CD40 and the antibody, including immunoblotting and immunoprecipitation assays. Such kits may further contain any one or more of the following: an antagonist, an additional anti-CD40 antibody, a pharmaceutical reference material, a protein purification column, an immunoglobulin affinity purification buffer, an assay diluent for cells, an instruction, or literature. The anti-CD40 antibody of the present disclosure may be used in different types of diagnostic tests, for example, in an in vitro or in vivo detection of various diseases, or the presence of drugs, toxins, or other proteins. For example, detection may be performed using serum or blood samples obtained from a subject to test related diseases. The kit may also be used in scientific research to detect human CD40 protein in a test sample. Such related diseases may include CD40-related diseases, such as cancer. Of course, the antibody or antigen-binding fragment thereof provided herein may also be used in radioimmunoassay and radioimmunotherapy of the above diseases. The binding molecule is also applicable to the above application scenarios and will not be described repeatedly.

The kit may further include conventional reagents used for CD40 detection, such as a coating buffer.

In a twelfth aspect, the present disclosure provides use of the antibody or antigen-binding fragment thereof according to the first aspect of the present disclosure, the nucleic acid molecule according to the second aspect of the present disclosure, the expression vector according to the third aspect of the present disclosure, or the recombinant cell according to the fifth aspect of the present disclosure in the preparation of a kit for detecting CD40. As described above, the antibody or antigen-binding fragment thereof according to some specific embodiments of the present disclosure is capable of effectively binding to human CD40 protein and blocking the interaction between CD40 protein and CD40L. Therefore, the antibody or antigen-binding fragment thereof can be used for the manufacture of a kit for detecting CD40 protein. The kit is capable of effective qualitative or quantitative detection of human CD40 protein.

In a thirteenth aspect, the present disclosure provides a method for treating and/or preventing a CD40-mediated disease. According to an embodiment of the present disclosure, the method includes: administering to a subject a pharmaceutically acceptable amount of at least one of: 1) the antibody or antigen-binding fragment thereof according to the first aspect of the present disclosure; 2) the nucleic acid molecule according to the second aspect of the present disclosure; 3) the expression vector according to the third aspect of the present disclosure; 4) the recombinant cell according to the fifth aspect of the present disclosure; 5) the immunoconjugate according to the sixth aspect of the present disclosure; 6) the composition according to the seventh aspect of the present disclosure; or 7) the medicament according to the eighth aspect of the present disclosure. As described above, the antibody or antigen-binding fragment thereof is capable of binding to human CD40 protein and effectively treating or preventing a CD40-mediated disease, preferably transplant rejection, an autoimmune disease, an infectious disease, or cancer. Therefore, the method according to embodiments of the present disclosure is capable of effectively treating or preventing a CD40-mediated disease.

According to an embodiment of the present disclosure, the above method for treating or preventing the disease may further include at least one of the following additional technical features.

According to an embodiment of the present disclosure, the autoimmune disease includes at least one of systemic lupus erythematosus, rheumatoid arthritis, systemic vasculitis, scleroderma, dermatomyositis, autoimmune hemolytic anemia, autoimmune thyroid disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, pemphigoid, primary biliary cirrhosis, multiple sclerosis, or acute idiopathic polyneuritis.

According to an embodiment of the present disclosure, the cancer includes at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head and neck cancer.

The effective amount of the antibody or antigen-binding fragment thereof or the pharmaceutical composition of the present disclosure may vary depending on factors such as the mode of administration and the severity of the disease to be treated. A preferred effective amount may be determined by those of ordinary skill in the art based on various factors (e.g., through clinical studies). Such factors include, but are not limited to, pharmacokinetic parameters of the active ingredient, such as bioavailability, metabolism, and half-life; the severity of the disease to be treated; the patient's body weight; the patient's immune status; and the route of administration. For example, depending on exigencies of the therapeutic situation, multiple divided doses may be administered daily, or the dosage may be proportionally adjusted.

In a fourteenth aspect, the present disclosure provides a method for detecting CD40. According to an embodiment of the present disclosure, the method includes: detecting a test sample by using the antibody or antigen-binding fragment thereof according to the first aspect of the present disclosure or the kit according to the eleventh aspect of the present disclosure. As described above, the aforementioned antibody or antigen-binding fragment thereof is capable of effectively binding to CD40. Therefore, the method of the present disclosure can effectively detect CD40, such as qualitatively or quantitatively detecting CD40 in a biological sample, and can also be applied to evaluate the status of an individual. For example, after obtaining the CD40 level of the individual, it is determined whether the CD40 level is higher or lower than a normal level. The biological sample may be cells, tissues, blood, etc.

According to an embodiment of the present disclosure, an immune complex is formed by the antibody or antigen-binding fragment thereof and CD40 in the test sample. Whether the immune complex is present is detected, and when the immune complex is present, it is determined that the test sample includes CD40.

According to an embodiment of the present disclosure, the presence of the immune complex indicates the presence of CD40 in the test sample.

In a fifteenth aspect, the present disclosure provides a method for inhibiting the growth of a cell expressing human CD40 antigen. The method includes: administering to a cell the antibody or antigen-binding fragment thereof according to the first aspect of the present disclosure. The antibody or antigen-binding fragment thereof specifically bindS to the CD40 antigen on the surface of human cells. The binding of the antibody or antigen-binding fragment thereof to the CD40 antigen inhibits growth or differentiation of the cell.

In a sixteenth aspect, the present disclosure provides a method for inducing depletion of peripheral B cells. The method includes: administering to cells the antibody or antigen-binding fragment thereof according to the first aspect of the present disclosure. The antibody or antigen-binding fragment thereof specifically binds to the CD40 antigen on the surface of human cells. The binding of the antibody or antigen-binding fragment thereof to the CD40 antigen induces depletion of the cells.

Additional aspects and advantages of the present disclosure will be provided at least in part in the following description, or will become apparent at least in part from the following description, or can be learned from practicing of the present disclosure.

### BRIEF DESCRIPTION OF THE DRAWINGS

The above and/or additional aspects and advantages of the present disclosure will become apparent and readily understood from the following description of embodiments in conjunction with the accompanying drawings.

FIG. 1 shows ELISA results demonstrating binding of the 21G6 antibody, the reference antibody Iscalimab, and the reference antibody Ravagalimab to human CD40 protein according to a specific embodiment of the present disclosure.

FIG. 2 shows ELISA results demonstrating binding of the 21G6 antibody, the reference antibody Iscalimab, and the reference antibody Ravagalimab to cynomolgus CD40 protein according to a specific embodiment of the present disclosure.

FIG. 3 shows flow cytometry results demonstrating binding of the 21G6 antibody, the reference antibody Iscalimab, and the reference antibody Ravagalimab to HCT-116 human colorectal cancer cells according to a specific embodiment of the present disclosure.

FIG. 4 shows flow cytometry results demonstrating binding of the 21G6 antibody and the humanized h21G6 antibody to HCT-116 human colorectal cancer cells according to a specific embodiment of the present disclosure.

FIG. 5 shows flow cytometry results demonstrating binding of the 21G6 antibody, the reference antibody Iscalimab, and the reference antibody Ravagalimab to human peripheral blood B cells according to a specific embodiment of the present disclosure.

FIG. 6 shows flow cytometry results demonstrating blocking the binding of CD40L-mFc to HCT-116 human colorectal cancer cells by the 21G6 antibody, the reference antibody Iscalimab, and the reference antibody Ravagalimab according to a specific embodiment of the present disclosure.

FIG. 7 shows flow cytometry results demonstrating blocking the binding of CD40L-mFc to HCT-116 human colorectal cancer cells by the 21G6 antibody and the humanized h21G6 antibody according to a specific embodiment of the present disclosure.

FIG. 8 shows results demonstrating inhibition of CD40 reporter cell activation mediated by CD40L-mFc by the 21G6 antibody, the reference antibody Iscalimab, and the reference antibody Ravagalimab according to a specific embodiment of the present disclosure.

FIG. 9 shows results demonstrating upregulation of CD86 expression on human peripheral blood B cells induced by the 21G6 antibody, the reference antibody Iscalimab, and the reference antibody Ravagalimab according to a specific embodiment of the present disclosure.

FIG. 10 shows results demonstrating inhibition of upregulation of CD80 expression on B cells mediated by CD40L-mFc by the 21G6 antibody, the reference antibody Iscalimab, and the reference antibody Ravagalimab according to a specific embodiment of the present disclosure.

FIG. 11 shows results demonstrating inhibition of upregulation of CD86 expression on B cells mediated by CD40L-mFc by the 21G6 antibody, the reference antibody Iscalimab, and the reference antibody Ravagalimab according to a specific embodiment of the present disclosure.

FIG. 12 shows results demonstrating inhibition of B-cell proliferation mediated by CD40L-mFc by the 21G6 antibody, the reference antibody Iscalimab, and the reference antibody Ravagalimab according to a specific embodiment of the present disclosure.

### DETAILED DESCRIPTION OF THE EMBODIMENTS

Specific embodiments of the present disclosure are described in detail below. It should be understood that the specific embodiments described herein are only used for illustration and explanation of the present disclosure and are not intended to limit the present disclosure.

It should be noted that the terms “first” and “second” are only used for descriptive purposes, and shall not be understood as indicating or implying relative importance, or as implicitly indicating the number of indicated technical features. Therefore, a feature defined with “first” and “second” may explicitly or implicitly include one or more such features. Further, in the description of the present disclosure, the term “plurality” or “more” means at least two, unless otherwise defined.

As used herein, the terms “comprises” or “includes” are open-ended expressions, meaning that they include the stated elements but do not exclude additional elements.

As used herein, the terms “optionally,” “optional,” or “option” generally mean that the subsequently described event or condition may but need not occur, and that the description encompasses both instances in which the event or condition occurs and instances in which the event or condition does not occur.

To facilitate understanding of the present disclosure, certain technical and scientific terms are specifically defined below. Unless otherwise expressly defined elsewhere herein, all other technical and scientific terms used herein have the meanings commonly understood by those of ordinary skill in the art to which the present disclosure belongs. Abbreviations for amino acid residues are the standard three-letter and/or one-letter codes used in the art to refer to one of the 20 commonly used L-amino acids.

The antibody or antigen-binding fragment of the present disclosure is typically prepared by biological synthesis methods. Based on the nucleotide sequences of the present disclosure, those skilled in the art can readily prepare the encoding nucleic acids of the present disclosure through various known methods. These methods include, but are not limited to, PCR, DNA artificial synthesis, etc. Specific methods can be referred to Molecular Cloning: A Laboratory Manual written by J. Sambrook. As an embodiment of the present disclosure, the encoding nucleic acid sequence of the present disclosure may be constructed by synthesizing nucleotide sequences separately and subsequently assembling them by overlap-extension PCR. The antibody or antigen-binding fragment thereof is numbered and defined according to the Kabat numbering system.

The antibody of the present disclosure includes a murine antibody, a chimeric antibody, and a humanized antibody, preferably a humanized antibody.

As used herein, the term “murine antibody” refers to a monoclonal antibody against human CD40 prepared according to common knowledge and skills in the art. During preparation, a test subject is injected with an antigen, and then hybridomas that express antibodies having the desired sequences or functional properties are isolated. In a preferred embodiment of the present disclosure, the murine CD40 antibody or antigen-binding fragment thereof may further includes a light chain constant region of a murine k chain, a murine 2 chain, or a variant thereof, or further includes a heavy chain constant region of murine IgG1, murine IgG2, murine IgG3, or a variant thereof.

The term “chimeric antibody” refers to an antibody formed by fusing the variable region of a murine antibody with the constant region of a human antibody, thereby reducing the immune response induced by the murine antibody. The variable region genes are cloned from mouse hybridoma cells, and then the constant region genes of the human antibody are cloned as needed. The mouse variable region genes and human constant region genes are linked to form a chimeric gene and inserted into an expression vector, and finally expressed in a eukaryotic expression system or prokaryotic expression system. In a preferred embodiment of the present disclosure, the antibody light chain of the CD40 chimeric antibody further includes a light chain Fc region of a humanized K chain, A chain, or a variant thereof. The antibody heavy chain of the CD40 chimeric antibody further includes a heavy chain constant region of humanized IgG1, IgG2, IgG3, IgG4, or a variant thereof.

The term “humanized antibody”, also known as “CDR-grafted antibody”, refers to an antibody produced by grafting mouse CDR sequences into a human antibody variable region framework, i.e., into framework sequences derived from different human germline antibody genes. Such framework sequences may be obtained from publicly available DNA databases or published references that include germline antibody gene sequences. For example, germline DNA sequences of human heavy variable region genes and human light chain variable region genes can be found in the “VBase” human germline sequence database. In order to avoid a decrease in activity while reducing immunogenicity, minimal reverse mutations or back mutations may be introduced into the human antibody variable region framework sequences to maintain the activity.

As used herein, the “monoclonal antibody” refers to an antibody with a single antigen-binding site.

As used herein, the “polyclonal antibody” refers to an antibody with two or more different antigen-binding sites.

As used herein, the terms “mutant” or “variant” may refer to a molecule obtained by introducing one or more nucleotides or amino acid mutations into any naturally occurring or engineered molecule.

The terms “complementary determining region”, “CDR”, or “CDR sequence” refer to amino acid sequences in an antibody that are responsible for antigen binding. For example, such regions generally include amino acid residues located approximately at positions 23-34 (L1), 50-56 (L2), and 89-97 (L3) in the light chain variable region, and positions 31-35B (H1), 50-65 (H2), and 95-102 (H3) in the heavy chain variable region according to Kabat et al., Sequences of Proteins of Immunological Interest, 5th Ed., Public Health Service, National Institutes of Health, Bethesda, Md. (1991); and/or the hypervariable loops including amino acid residues located approximately at positions 26-32 (L1), 50-52 (L2), and 91-96 (L3) in the light chain variable region, and positions 26-32 (H1), 53-55 (H2), and 96-101 (H3) in the heavy chain variable region according to Chothia and Lesk, J. Mol. Biol. 196:901-917 (1987).

As used herein, the term “identity” when used with respect to an amino acid sequence or nucleic acid sequence relative to a reference sequence, refers to the percentage of identical amino acids or nucleotides between two amino acid sequences or nucleic acid sequences as determined by conventional methods. See, for example, Ausubel et al. (eds.), Current Protocols in Molecular Biology, Chapter 19 (1995); and the ALIGN program (Dayhoff, Atlas of Protein Sequence and Structure 5, Suppl. 3 (1978)). Numerous algorithms are available for sequence alignment and identity determination, including the homology alignment algorithm of Needleman et al. (1970), J. Mol. Biol. 48:443; the local homology algorithm of Smith et al. (1981), Adv. Appl. Math. 2:482; the similarity search method of Pearson et al. (1988), Proc. Natl. Acad. Sci. USA 85:2444; the Smith-Waterman algorithm (Methods Mol. Biol. 70:173-187 (1997)); and the BLASTP, BLASTN, and BLASTX algorithms described by Altschul et al. (1990), J. Mol. Biol. 215:403-410. Computer programs implementing these algorithms are commercially available and include, but are not limited to, ALIGN or MegAlign (DNASTAR), WU-BLAST-2 (Altschul et al., Methods Enzymol. 266:460-480 (1996)), GAP, BESTFIT, BLAST, FASTA, and TFASTA available in Version 8 of the Genetics Computer Group (GCG) software package (Madison, Wisconsin, USA), and CLUSTAL available in the PC/Gene software package from Intelligenetics (Mountain View, California, USA).

Without substantially affecting antibody activity (remaining at least 95% of activity), substitution, one or more amino acids (e.g., 1, 2, 3, 4, 5, 6, 7, 8, 9, 10, or more amino acids) may be substituted, added, and/or deleted by those skilled in the art to obtain variants of the antibody or functional fragment sequence. All such variants are considered to be encompassed by the present disclosure. For example, amino acids having similar properties may be substituted within the variable region. The variant sequences of the present disclosure may have at least 80%, 85%, 90%, 95%, 96%, 97%, 98%, or 99% identity (or homology) to the reference sequence. Sequence identity may be determined using sequence analysis software, such as the BLAST program, particularly BLASTP or TBLASTN, with default parameters. Unless otherwise indicated, all amino acid sequences described herein are presented in the N-terminal to C-terminal orientation.

As mentioned above, the monoclonal antibody according to the present disclosure may be a full-length antibody or may include only functional fragments thereof (e.g., Fab, F(ab′) 2, or scFv fragments), or may be modified to affect functionality. The present disclosure also encompasses an anti-CD40 antibody with modified glycosylation patterns. In some applications, it may be desirable to remove undesired glycosylation sites or to provide antibodies lacking fucose residues in oligosaccharide chains, for example, to enhance antibody-dependent cellular cytotoxicity (ADCC). In other applications, galactosylation modifications may be performed to alter complement-dependent cytotoxicity (CDC).

As used herein, the “full-length antibody” refers to a tetrapeptide-chain structure formed by linking two identical light chains and two identical heavy chains via interchain disulfide bonds, such as immunoglobulin G (IgG), immunoglobulin A (IgA), immunoglobulin M (IgM), immunoglobulin D (IgD) or immunoglobulin E (IgE). Immunoglobulin of the same class may be further divided into different subclasses according to amino acid compositions, such as IgG1, IgG2, IgG3, and IgG4. Immunoglobulin light chains are divided into K chain or A chain according to differences in their constant regions.

As used herein, the term “functional fragment” particularly refers to an antibody fragment, such as a CDR-grafted antibody, Fab, Fab′, (Fab′) 2, Fv, scFv, a nanobody, or any fragment chemically modified or incorporated into liposomes to increase half-life. Such chemical modifications include, for example, addition of poly(alkylene) glycol, such as polyethylene glycol (“PEGylation”), resulting in PEGylated fragments designated Fv-PEG, scFv-PEG, Fab-PEG, (Fab′) 2-PEG, or Fab′-PEG. These fragments retain CD40-binding activity. Preferably, the functional fragment consists of or includes a partial sequence of the heavy or light chain variable region of the parent antibody. The partial sequence is sufficient to retain identical binding specificity and adequate affinity as the parent antibody. For CD40, the binding specificity or affinity is preferably at least 1/100, more preferably at least 1/10, of the parent antibody. Such functional fragments generally include at least 3 amino acids and preferably include 5, 10, 15, 25, 50, or 100 consecutive amino acids derived from the parent antibody sequence.

As used herein, unless contrarily stated, the term “antigen-binding fragment” generally refers to an antigen-binding antibody fragment that include a portion of a complete antibody, generally an antigen-binding region or a variable region. Exemplary antigen-binding fragments include a CDR-grafted antibody, Fab, Fab′, F(ab′) 2, Fv, scFv, nanobody, and the like.

As used herein, the term “CDR-grafted antibody” refers to an antibody in which the CDRs of a monoclonal antibody from one species are grafted into the variable region of an antibody from another species. For example, the CDRs of a murine monoclonal antibody may be grafted into the variable region of a human antibody to replace the CDRs of the human antibody, thereby conferring the antigen-binding specificity of the murine monoclonal antibody on the human antibody while reducing heterologous immunogenicity.

As used herein, the term “Fab antibody” or “Fab” generally refers to an antibody fragment including only a Fab molecule, which is composed of VH and CH1 of a heavy chain and a complete light chain, with the light chain and the heavy chain linked through a disulfide bond.

As used herein, the term “nanobody” (single-domain antibody or VHH antibody) is originally described as an antigen-binding immunoglobulin (variable) domain of a “heavy chain antibody” (that is, “an antibody lacking a light chain”) (Hamers-Casterman C, AtarhouchT, Muyldermans S, Robinson G, Hamers C, Songa E B, Bendahman N, Hamers R.: “Naturally occurring antibodies devoid of light chains”; Nature 363, 446-448 (1993)). Such antibody only includes a heavy chain variable region (VH) and conventional CH2 and CH3 regions, and binds specifically to an antigen through the heavy chain variable region.

As used herein, the term “Fv antibody” generally refers to an antibody fragment formed by linking a light chain variable region (VL) and a heavy chain variable region (VH) through non-covalent bonds, representing the smallest functional fragment of an antibody molecule that retains the complete antigen binding site.

As used herein, the term “single-chain antibody” or “scFv” refers to a fragment formed by linking a heavy chain variable region and a light chain variable region of the antibody through a short peptide linker.

The amino acid or nucleic acid sequences involved in the present disclosure are specifically listed in Table 1.

TABLE 1

| SEQ | | |
|---|---|---|
| ID | | |
| NO: | Sequence | Description |
| 1 | GYTFTSFT | Heavy chain |
| | | variable |
| | | region |
| | | HCDR1 of |
| | | 21G6 |
| | | antibody |
| 2 | INPNSGNT | Heavy chain |
| | | variable |
| | | region |
| | | HCDR2 of |
| | | 21G6 |
| | | antibody |
| 3 | TRWGAGFDY | Heavy chain |
| | | variable |
| | | region |
| | | HCDR3 of |
| | | 21G6 |
| | | antibody |
| 4 | SSVSY | Light chain |
| | | variable |
| | | region |
| | | LCDR1 of |
| | | 21G6 |
| | | antibody |
| 5 | RTS | Light chain |
| | | variable |
| | | region |
| | | LCDR2 of |
| | | 21G6 |
| | | antibody |
| 6 | LQYHTYPLT | Light chain |
| | | variable |
| | | region |
| | | LCDR3 of |
| | | 21G6 |
| | | antibody |
| 7 | QVQLRQSGAELARPGASVKMSCKASGYTFTSFTIHWVIQRP | Heavy chain |
| | GQGLEWIGYINPNSGNTNYNQKFKDKATLTADKSSSTAYIQ | variable |
| | LNSLTSEDSAVYFCTRWGAGFDYWGQGTTLTVSS | region of |
| | | 21G6 |
| | | antibody |
| 8 | QIVLTQSPAIMSASPGEKVTISCSASSSVSYVYWYQQKLGSS | Light chain |
| | PKPWISRTSNLASGVPARFSGSGSGTSYSLTISSMEAEDAVT | variable |
| | YYCLQYHTYPLTFGGGTKLEIK | region of |
| | | 21G6 |
| | | antibody |
| 9 | QVQLRQSGAELARPGASVKMSCKASGYTFTSFTIHWVIQRP | Heavy chain |
| | GQGLEWIGYINPNSGNTNYNQKFKDKATLTADKSSSTAYIQ | of 21G6 |
| | LNSLTSEDSAVYFCTRWGAGFDYWGQGTT | antibody |
| | LTVSSASTKGPSVFPLAPSSKSTSGGTAALGCLVKDYFPEPV | |
| | TVSWNSGALTSGVHTFPAVLQSSGLYSLSSVVTVPSSSLGTQ | |
| | TYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSV | |
| | LTVLHQDWLNGKEYKCKVSNKALPAPIEKT | |
| | ISKAKGQPREPQVYTLPPSREEMTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSPGK | |
| 10 | QIVLTQSPAIMSASPGEKVTISCSASSSVSYVYWYQQKLGSS | Light chain |
| | PKPWISRTSNLASGVPARFSGSGSGTSYSLTISSMEAEDAVT | of 21G6 |
| | YYCLQYHTYPLTFGGGTKLEIK | antibody |
| | RTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |
| 11 | QVQLVQSGAEVKRPGASVKVSCKASGYTFTSFTIHWVRQA | Heavy chain |
| | PGQGLEWMGYINPNSGNTNYNQKFKDRVTLTADKSTSTAY | variable |
| | MELNSLTSEDTAVYFCARWGAGFDYWGQGTLVTVSS | region of |
| | | h21G6 |
| | | antibody |
| 12 | EIVLTQSPAILSVSPGEKVTISCSASSSVSYVYWYQQKPGSA | Light chain |
| | PKLLISRTSNLASGVPARFSGSGSGTDFSLTISSLQAEDAATY | variable |
| | YCLQYHTYPLTFGGGTKLEIK | region of |
| | | h21G6 |
| | | antibody |
| 13 | QVQLVQSGAEVKRPGASVKVSCKASGYTFTSFTIHWVRQA | Heavy chain |
| | PGQGLEWMGYINPNSGNTNYNQKFKDRVTLTADKSTSTAY | of h21G6 |
| | MELNSLTSEDTAVYFCARWGAGFDYWGQGTL | antibody |
| | VTVSSASTKGPSVFPLAPSSKSTSGGTAALGCLVKDYFPEPV | |
| | TVSWNSGALTSGVHTFPAVLQSSGLYSLSSVVTVPSSSLGTQ | |
| | TYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSV | |
| | LTVLHQDWLNGKEYKCKVSNKALPAPIEKT | |
| | ISKAKGQPREPQVYTLPPSREEMTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSPGK | |
| 14 | EIVLTQSPAILSVSPGEKVTISCSASSSVSYVYWYQQKPGSA | Light chain |
| | PKLLISRTSNLASGVPARFSGSGSGTDFSLTISSLQAEDAATY | of h21G6 |
| | YCLQYHTYPLTFGGGTKLEIK | antibody |
| | RTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |
| 15 | QVQLVESGGGVVQPGRSLRLSCAASGFTFSSYGMHWVRQ | Heavy chain |
| | APGKGLEWVAVISYEESNRYHADSVKGRFTISRDNSKITLYL | of Iscalimab |
| | QMNSLRTEDTAVYYCARDGGIAAPGPDYWGQ | |
| | GTLVTVSSASTKGPSVFPLAPSSKSTSGGTAALGCLVKDYFP | |
| | EPVTVSWNSGALTSGVHTFPAVLQSSGLYSLSSVVTVPSSSL | |
| | GTQTYICNVNHKPSNTKVDKKVEPKSCDK | |
| | THTCPPCPAPEAAGGPSVFLFPPKPKDTLMISRTPEVTCVVV | |
| | DVSHEDPEVKFNWYVDGVEVHNAKTKPREEQYNSTYRVV | |
| | SVLTVLHQDWLNGKEYKCKVSNKALPAPIEK | |
| | TISKAKGQPREPQVYTLPPSREEMTKNQVSLTCLVKGFYPS | |
| | DIAVEWESNGQPENNYKTTPPVLDSDGSFFLYSKLTVDKSR | |
| | WQQGNVFSCSVMHEALHNHYTQKSLSLSPGK | |
| 16 | DIVMTQSPLSLTVTPGEPASISCRSSQSLLYSNGYNYLDWYL | Light chain |
| | QKPGQSPQVLISLGSNRASGVPDRFSGSGSGTDFTLKISRVE | of Iscalimab |
| | AEDVGVYYCMQARQTPFTFGPGTKV | |
| | DIRRTVAAPSVFIFPPSDEQLKSGTASVVCLLNNFYPREAKV | |
| | QWKVDNALQSGNSQESVTEQDSKDSTYSLSSTLTLSKADY | |
| | EKHKVYACEVTHQGLSSPVTKSFNRGEC | |
| 17 | EVQLVESGGGLVKPGGSLRLSCAASGFTFSDYGMNWVRQA | Heavy chain |
| | PGKGLEWIAYISSGRGNIYYADTVKGRFTISRDNAKNSLYL | of |
| | QMNSLRAEDTAVYYCARSWGYFDVWGQGTT | Ravagalimab |
| | VTVSSASTKGPSVFPLAPSSKSTSGGTAALGCLVKDYFPEPV | |
| | TVSWNSGALTSGVHTFPAVLQSSGLYSLSSVVTVPSSSLGTQ | |
| | TYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSV | |
| | LTVLHQDWLNGKEYKCKVSNKALPAPIEKT | |
| | ISKAKGQPREPQVYTLPPSREEMTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSPGK | |
| 18 | DIVMTQSPDSLAVSLGERATINCKSSQSLLNRGNQKNYLTW | Light chain |
| | FQQKPGQPPKLLIYWASTRESGVPDRFSGSGSGTDFTLTISSL | of |
| | QAEDVAVYYCQNDYTYPLTFGQGTKL | Ravagalimab |
| | EIKRTVAAPSVFIFPPSDEQLKSGTASVVCLLNNFYPREAKV | |
| | QWKVDNALQSGNSQESVTEQDSKDSTYSLSSTLTLSKADY | |
| | EKHKVYACEVTHQGLSSPVTKSFNRGEC | |
| 19 | MQKGDQNPQIAAHVISEASSKTTSVLQWAEKGYYTMSNNL | CD40L-mFc |
| | VTLENGKQLTVKRQGLYYIYAQVTFCSNREASSQAPFIASL | |
| | CLKSPGRFERILLRAANTHSSAKPCGQQSIHLGGVFELQPG | |
| | ASVFVNVTDPSQVSHGTGFTSFGLLKLGGGGSGGGGSGGG | |
| | GSGGGGSMQKGDQNPQIAA | |
| | HVISEASSKTTSVLQWAEKGYYTMSNNLVTLENGKQLTVK | |
| | RQGLYYIYAQVTFCSNREASSQAPFIASLCLKSPGRFERILLR | |
| | AANTHSSAKPCGQQSIHLGGVFELQPGASVFVNVTDPSQVS | |
| | HGTGFTSFGLLKLGGGGSGGGGSGGGGSGGGGSMQKGDQ | |
| | NPQIAAHVISEASSKTTS | |
| | VLQWAEKGYYTMSNNLVTLENGKQLTVKRQGLYYIYAQV | |
| | TFCSNREASSQAPFIASLCLKSPGRFERILLRAANTHSSAKPC | |
| | GQQSIHLGGVFELQPGASVFVNVTDPSQVSHGTGFTSFGLL | |
| | KLGGGGSEPRGPTIKPCPPCKCPAPNLLGGPSVFIFPPKIKDV | |
| | LMISLSPIVTCVVVD | |
| | VSEDDPDVQISWFVNNVEVHTAQTQTHREDYNSTLRVVSA | |
| | LPIQHQDWMSGKEFKCKVNNKDLPAPIERTISKPKGSVRAP | |
| | QVYVLPPPEEEMTKKQVTLTCMVTDFMPEDIYVEWTNNG | |
| | KTELNYKNTEPVLDSDGSYFMYSKLRVEKKNWVERNSYS | |
| | CSVVHEGLHNHHTTKSFSRTPGK | |
| 20 | MVRLPLQCVLWGCLLTAVHPEPPTACREKQYLINSQCC | CD40 |
| | SLCQPGQKLVSDCTEFTETECLPCGESEFLDTWNRETHCHQ | |
| | HKYCDPNLGLRVQQKGTSETDTICTCEEGWHCTSEACESC | |
| | VLHRSCSPGFGVKQIATGV | |
| | SDTICEPCPVGFFSNVSSAFEKCHPWTSCETKDLVVQQAGT | |
| | NKTDVVCGPQDRLRALVVIPIIFGILFAILLVLVFIKKVAKKP | |
| | TNKAPHPKQEPQEINFPDDLPGSNTAAPVQETLHGCQPVTQ | |
| | EDGKESRISVQERQ | |

Embodiments of the present disclosure are described in detail below. The embodiments described below are illustrative, and are merely used for explaining the present disclosure, and shall not be understood as limiting the present disclosure. Where specific techniques or conditions are not specified in the examples, they are conducted according to techniques or conditions described in the literature in the art or according to the product manual. Reagents or instruments used without indicating manufacturers are all conventional products commercially available in the market.

### Example 1: Preparation of Antibody

### (1) Generation of Murine Monoclonal Antibody Against Human CD40

The purified recombinant CD40 extracellular region His-tag fusion protein (CD40-His) (purchased from Acro) was used as the antigen to immunize the Balb/c mice (9 weeks old, purchased from Shanghai SLAC, weighing about 20 g).

The Balb/c mice were immunized three times with the purified antigen, complete Freund's adjuvant, and incomplete Freund's adjuvant, and immune responses were evaluated after collecting blood samples from the tail vein. Serum samples were screened by ELISA and flow cytometry to identify mice producing anti-human CD40 immunoglobulins. Splenocytes were isolated from the mouse with the highest anti-CD40 immunoglobulin titer and fused with mouse myeloma SP2/0 cells (ATCC No. CRL-1581). The fused hybridoma cells were subjected to antibody screening to obtain a murine monoclonal antibody.

Candidate hybridoma cells was cultured to a total cell number of 10⁶ cells. The cells were collected by centrifugation at 800 rpm for 10 minutes, and total RNA was extracted using a Trizol kit (purchased from Invitrogen). The total RNA was used as a template for reverse transcription to synthesize a cDNA library. The cDNA was then used as a template for PCR amplification of the nucleic acid sequences encoding the variable regions corresponding to the hybridoma cells. The primer sequences used in the PCR amplification reaction were complementary to the first framework region of the antibody variable region or the signal peptide region and the constant region (specific sequences were selected with reference to Larrick, J. W., et al., (1990) Scand. J. Immunol., 32, 121-128 and Coloma, J. J. et al., (1991) BioTechniques, 11, 152-156). PCR amplification was carried out in a 50 μL reaction system containing 2 μL of cDNA, 5 μL of 10×PCR buffer, 2 μL of each of upstream and downstream primers (5 μM), 2 μL of dNTPs, 1 μL of Taq enzyme (Takara, Ex Taq), and 38 μL of H₂O. Following pre-denaturation at 95 °C for 5 minutes, PCR amplification was carried out under the following cycling conditions: denaturation at 94 °C for 30 s, annealing at 58 °C for 45 s, and extension at 72 °C for 50 s for a total of 32 cycles, followed by a final extension at 72 °C for 7 minutes. After sequencing the amplified product, the sequences of the heavy chain variable region (SEQ ID NO: 7) and the light chain variable region (SEQ ID NO: 8) of the murine monoclonal antibody 21G6 were obtained.

### (2) Humanization of the Murine Antibody:

Based on the light chain variable region sequence and heavy chain variable region sequence of the anti-CD40 antibody 21G6 antibody, a human framework template exhibiting the highest homology in the non-CDR regions was selected. The CDRs of the murine antibody were grafted onto the selected human framework template to replace the CDRs of the human template, thereby generating a humanized antibody. Subsequently, based on the three-dimensional structure of the murine antibody, back mutations were introduced at buried residues, residues that directly interact with the CDRs, and residues that exert critical effects on the conformation of VL and VH. The resulting humanized antibody was designated h21G6. The heavy chain variable region sequence of the humanized antibody h21G6 is as set forth in SEQ ID NO: 11, and the light chain variable region sequence is as set forth in SEQ ID NO: 12.

### Example 2: Production of Antibody

The antibodies were produced according to the following procedure: (1) ExpiCHO cells (purchased from Thermo Fisher) were cultured in ExpiCHO Expression Medium (purchased from Thermo Fisher), and the cell density was adjusted to 6×10⁶ cells/mL to obtain an ExpiCHO cell suspension. (2) pcDNA3.4 vectors containing the antibody heavy chain and light chain (synthesized by Nanjing GenScript) were added at a ratio of 1:1 into 2 mL OptiSFM medium (purchased from Thermo Fisher) to obtain solution A. (3) 160 μL ExpiFectamine CHO transfection reagent (purchased from Thermo Fisher) was added to 2 mL of OptiSFM medium (purchased from Thermo Fisher) to obtain solution B. (4) Solution A was mixed with solution B to obtain a transfection mixture, and the entire transfection mixture was added to 50 mL of the ExpiCHO cell suspension within 5 minutes. (5) After incubation at conditions of 37 °C and 5% CO₂ for 1 day, 8 mL of Feed and 300 μL of Enhancer (purchased from Thermo Fisher). The culture was then transferred to conditions of 32 °C and 5% CO₂. On day 5, an additional 8 mL Feed was added. After a total culture period of 9 days, the culture supernatant was harvested. (6) Affinity purification was performed on the culture supernatant by utilizing a Protein A purification column (purchased from Nano-micro) to obtain the target antibody.

### Example 3: ELISA Binding Assay of 21G6 Antibody

ELISA assay was performed to evaluate the binding properties of the CD40 antibody. The CD40 extracellular region His-tag fusion proteins were coated onto a 96-well plate, and the signal intensity after antibody addition was used to evaluate the binding properties of the antibodies to CD40 protein.

Human CD40-His protein or cynomolgus CD40-His protein (purchased from Acro) was diluted to a concentration of 2 μg/ml in PBS buffer and added to a 96-well plate at 100 μl/well. The plate was placed at 4 °C overnight. The PBS buffer in the 96-well plate was removed. The plate was washed 6 times with PBST buffer (PBS containing 0.1% Tween 20, pH 7.2), and then 200 μl/well PBS/10% BSA was added. The plate was incubated at 37 °C for 2 hours for blocking. The blocking solution was removed, and the plate was washed 6 times with PBST. CD40 antibody 21G6 (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), and hIgG1LALA (purchased from Biointron) were serially diluted with PBST/0.05% BSA and added at 100 μl/well. The plate was incubated at 37 °C for 1 hour. The reaction system was removed, and the plate was washed 6 times with PBST. An HRP (horseradish peroxidase)-labeled anti-human IgG secondary antibody (purchased from Jacksonlab) diluted in PBST/0.05% BSA was added at 100 μl/well. The plate was incubated at 37 °C for 1 hour. After the plate was washed 6 times with PBST, 80 μl/well of TMB (tetramethylbenzidine) was added, and the plate was incubated at room temperature for 3 minutes. The reaction was terminated by adding 80 μl/well of 4M sulfuric acid. The absorbance was read at 450 nm using a microplate reader.

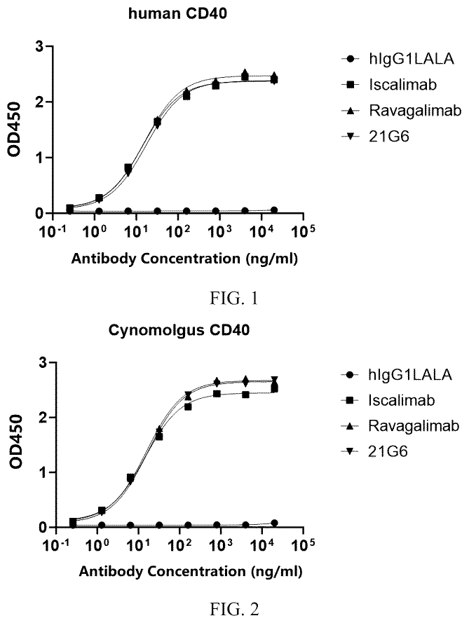

The experimental results shown in FIG. 1 and FIG. 2 indicate that the CD40 antibody of the present disclosure binds not only to human CD40 protein but also to cynomolgus CD40 protein and exhibits binding activities comparable to those of the reference antibodies Iscalimab and Ravagalimab

### Example 4: Flow Cytometry Binding Assay of 21G6 Antibody

Flow cytometry assay was used to evaluate the binding properties of the CD40 antibodies. The CD40 antibodies was added to cells, and the signal intensity after antibody addition was used to determine the binding properties of the murine antibody to CD40.

(1) HCT-116 human colorectal cancer cells were diluted to 2×10⁶ cells/ml in PBS and added into 1.5 ml EP tubes at 100 μl/tube. Goat serum was added at 10 μl/tube, and the cells were incubated at 4 °C for 30 minutes for blocking. Different concentrations of 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), humanized h21G6 antibody (having a heavy chain sequence as set forth in SEQ ID NO: 13 and a light chain sequence as set forth in SEQ ID NO: 14), and hIgG1LALA (purchased from Biointron) were added, and the cells were incubated at 4 °C for 30 minutes. 1 ml of PBS was added to each EP tube, followed by centrifugation at 3,500 rpm for 5 minutes at 4 °C. The supernatant was discarded, and the cells were washed once with PBS. After centrifugation, the supernatant was discarded, and the cells were resuspended in 100 μl/tube of PBS, followed by the addition of 1 μl/tube of APC-labeled goat anti-human IgG secondary antibody (purchased from Jackson Lab). The cells were incubated at 4 °C in the dark for 30 minutes. The cells were washed twice with PBS, and the supernatant was discarded after centrifugation. The cells were resuspended in 200 μl/tube of PBS and analyzed using a flow cytometry.

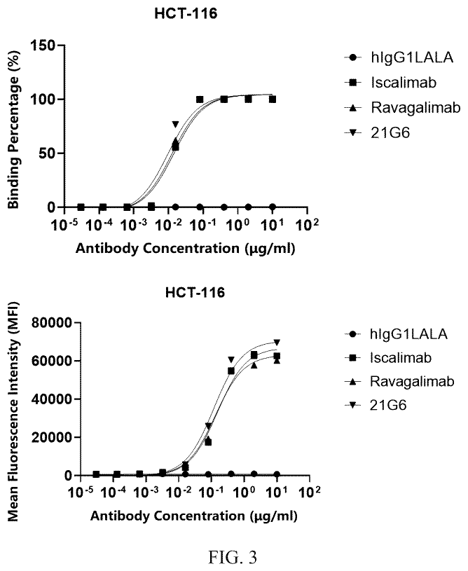

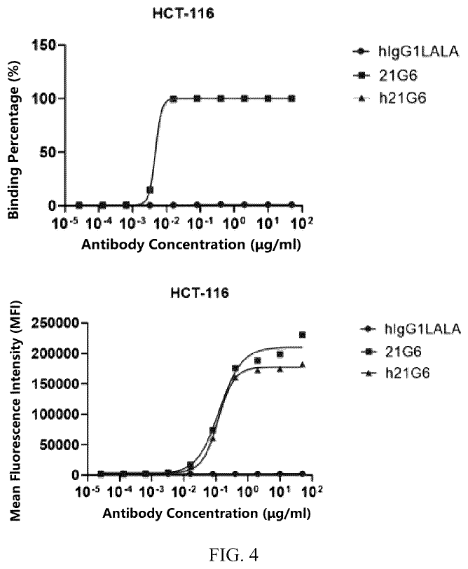

The experimental results shown in FIG. 3 demonstrate that the 21G6 antibody of the present disclosure binds to HCT-116 human colorectal cancer cells and exhibits stronger binding activity than the reference antibodies Iscalimab and Ravagalimab. The results of the binding activity of the humanized h21G6 antibody against HCT-116 human colorectal cancer cells are shown in FIG. 4, further demonstrating that the humanized h21G6 antibody of the present disclosure binds to HCT-116 human colorectal cancer cells and exhibits binding activity comparable to that of the 21G6 antibody.

(2) Human peripheral blood mononuclear cells (PBMCs, purchased from HYS-BIO) were diluted to 2×10⁶ cells/ml in PBS and added into 1.5 ml EP tubes at 100 μl/tube. Goat serum was added at 10 μl/tube, and the cells were incubated at 4 °C for 30 minutes for blocking. Different concentrations of 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), and hIgG1LALA (purchased from Biointron) were added, and the cells were incubated at 4 °C for 30 minutes. 1 ml of PBS was added to each EP tube, followed by centrifugation at 3,500 rpm for 5 minutes at 4 °C. The supernatant was discarded, and the cells were washed once with PBS. After centrifugation, the supernatant was discarded, and the cells were resuspended in 100 μl/tube of PBS, followed by the addition of 1 μl/tube of APC-labeled goat anti-human IgG secondary antibody (purchased from Jackson Lab) and FITC-labeled anti-CD19 antibody (purchased from BioLegend). The cells were incubated at 4 °C in the dark for 30 minutes. The cells were washed twice with PBS, and the supernatant was discarded after centrifugation. The cells were resuspended in 200 μl/tube of PBS and analyzed using a flow cytometry.

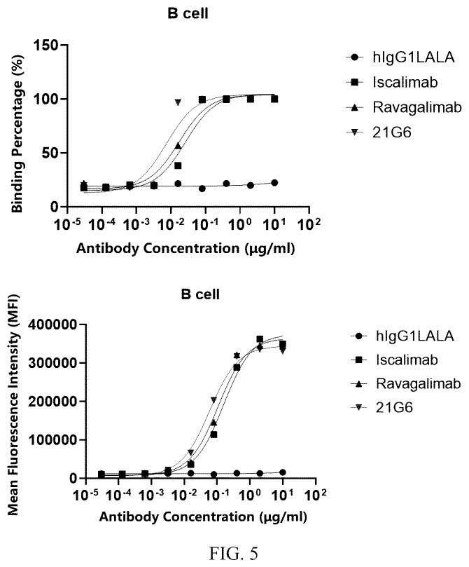

The experimental results shown in FIG. 5 further demonstrate that the 21G6 antibody of the present disclosure binds to human peripheral blood B cells and exhibits stronger binding activity than the reference antibodies Iscalimab and Ravagalimab.

### Example 5: Evaluation of Blocking Activity of CD40 Antibody

Flow cytometry assay was used to evaluate the blocking properties of the CD40 antibodies. The CD40 antibody and CD40L-mFc protein were added to HCT-116 cells, and the signal intensity after addition of an APC-labeled anti-mouse IgG antibody was used to determine the effect of the antibody on the binding of CD40L-mFc to HCT-116 cells.

HCT-116 human colorectal cancer cells were diluted to 2×10⁶ cells/ml in PBS and added into 1.5 ml EP tubes at 100 μl/tube. Goat serum was added at 10 μl/tube, and the cells were incubated at 4 °C for 30 minutes for blocking. Different concentrations of 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), and hIgG1LALA (purchased from Biointron) were added together with 0.5 μg/mL CD40L-mFc protein (having an amino acid sequence set forth in SEQ ID NO: 19), and the cells were incubated at 4 °C for 30 minutes. 1 ml of PBS was added to each EP tube, followed by centrifugation at 3,500 rpm for 5 minutes at 4 °C. The supernatant was discarded, and the cells were washed once with PBS. After centrifugation, the supernatant was discarded, and the cells were resuspended in 100 μl/tube of PBS, followed by the addition of 1 μl/tube of APC-labeled goat anti-mouse IgG secondary antibody (purchased from BioLegend). The cells were incubated at 4 °C in the dark for 30 minutes. The cells were washed twice with PBS, and the supernatant was discarded after centrifugation. The cells were resuspended in 200 μl/tube of PBS and analyzed using a flow cytometry.

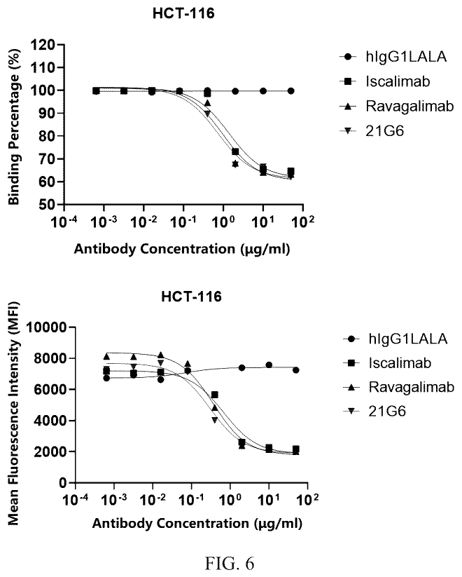

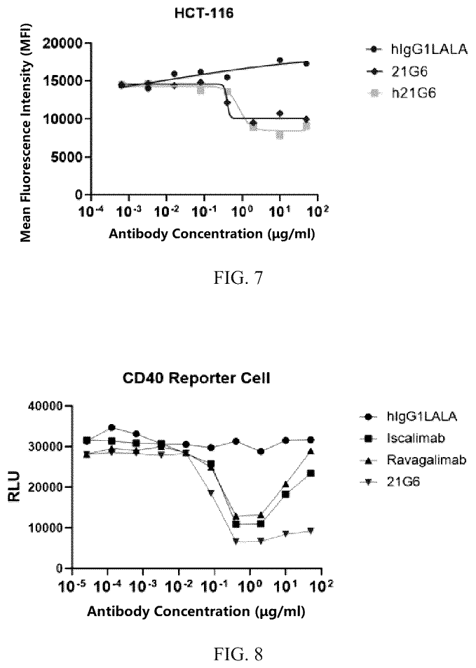

The experimental results shown in FIG. 6 and FIG. 7 demonstrate that the 21G6 antibody of the present disclosure blocks the binding of CD40L to HCT-116 cells and exhibits blocking activity comparable to that of the reference antibodies Iscalimab and Ravagalimab; and the humanized h21G6 antibody also blocks the binding of CD40L to HCT-116 cells and exhibits blocking activity comparable to that of the 21G6 antibody.

### Example 6: Inhibition of CD40 Reporter Cell Activation by 21G6 Antibody

Reporter cell assay was used to evaluate the effect of the CD40 antibody on CD40 downstream activation signals. CD40L-mFc and the CD40 antibody were added to CD40 reporter cells, and the luciferase signal intensity after antibody addition was used to determine the effect of the CD40 antibody on the CD40 downstream activation signals.

CD40 reporter cells (purchased from Cobioer Biosciences) were diluted to 2×10⁵ cells/ml in complete DMEM medium and added to a 96-well plate at 100 μl/well. 0.5 μg/ml of CD40L-mFc was added together with serially diluted CD40 antibody 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), and hIgG1LALA (purchased from Biointron). The cells were incubated at 37 °C for 6 hours. A luciferase substrate was added to the cells for measurement using a microplate reader.

The experimental results shown in FIG. 8 demonstrate that the CD40 antibody 21G6 of the present disclosure inhibits the activation of CD40 reporter cells. In contrast, the reference antibodies Iscalimab and Ravagalimab fail to achieve a comparable inhibitory effect, exhibiting inhibitory activity only at certain concentrations and with substantially weaker inhibition than antibody 21G6.

### Example 7: Effect of CD40 Antibody on B-Cell Activation

Flow cytometry assay was used to evaluate the effect of the CD40 antibody on CD86 expression on B cells. The CD40 antibody was added to human peripheral blood PBMCs, and the expression level of CD86 after antibody addition was used to determine the activation of B cells induced by the CD40 antibody.

Human peripheral blood PBMCs (purchased from HYS-BIO) were diluted to 2×10⁶ cells/ml in complete RPMI 1640 medium and added into a 96-well plate at 100 μL/well. CD40 antibody 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), and hIgG1LALA (purchased from Biointron) were added, and the cells were incubated at 37 °C for 24 hours. Fluorescently labeled anti-CD19 and anti-CD86 antibodies (purchased from BioLegend) were added. The cells were then resuspended in 200 μl/tube of PBS and analyzed by flow cytometry.

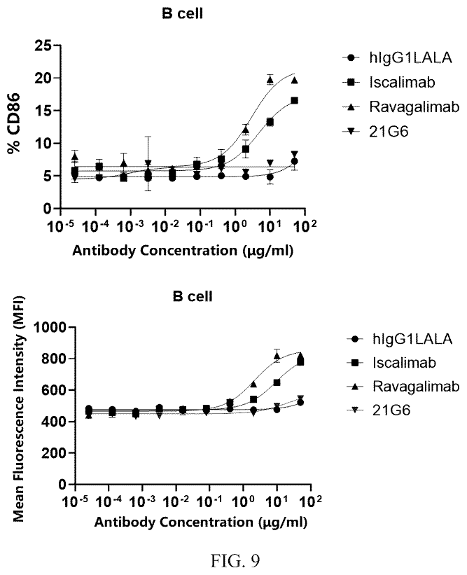

The experimental results shown in FIG. 9 demonstrate that the CD40 antibody 21G6 of the present disclosure does not promote CD86 expression on B cells, whereas the reference antibodies Iscalimab and Ravagalimab induce upregulation of CD86 expression on B cells. Although both 21G6 and the reference antibodies were developed as CD40 antagonists, activation of B cells by the reference antibodies is undesirable for the intended efficacy.

### Example 8: Inhibition of CD40L-Mediated B-Cell Activation by CD40 Antibody

Flow cytometry assay was used to evaluate the effect of the CD40 antibody on CD40L-mediated upregulation of CD80 and CD86 in B cells. CD40L-mFc and the CD40 antibody were added to human peripheral blood PBMCs, and the expression levels of CD80 and CD86 after antibody addition was used to determine the inhibitory effect of the CD40 antibody on CD40L-mediated B-cell activation.

Human peripheral blood PBMCs (purchased from HYS-BIO) were diluted to 2×10⁶ cells/ml in complete RPMI 1640 medium and added into a 96-well plate at 100 μL/well. CD40 antibody 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), the reference antibody Ravagalimab (having a heavy chain sequence as set forth in SEQ ID NO: 17 and a light chain sequence as set forth in SEQ ID NO: 18), and hIgG1LALA (purchased from Biointron) were added together with 0.5 μg/mL CD40L-mFc, and the cells were incubated at 37 °C for 24 hours. Fluorescently labeled anti-CD19, anti-CD80, and anti-CD86 antibodies (purchased from BioLegend) were added. The cells were then resuspended in 200 μl/tube of PBS and analyzed by flow cytometry.

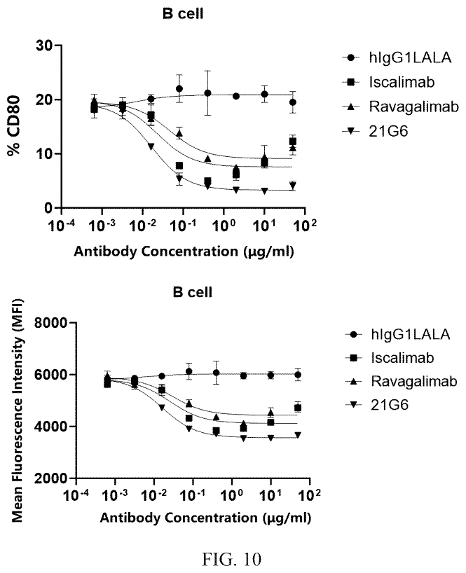

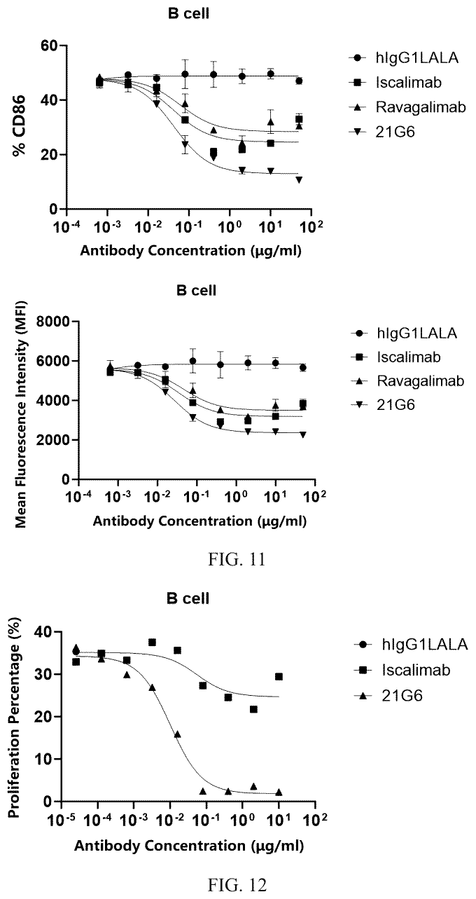

The experimental results are shown in FIG. 10 and FIG. 11. FIG. 10 demonstrates that the CD40 antibody 21G6 of the present disclosure inhibits the expression of CD80 on B cells and exhibits a stronger inhibitory effect than the reference antibodies Iscalimab and Ravagalimab, whose inhibitory effects decrease further at higher concentrations. FIG. 11 demonstrates that the CD40 antibody 21G6 of the present disclosure inhibits the expression of CD86 on B cells and exhibits a stronger inhibitory effect than the reference antibodies Iscalimab and Ravagalimab, whose inhibitory effects likewise decrease at high concentrations.

### Example 9: Inhibition of B-Cell Proliferation by CD40 Antibody

Flow cytometry assay was used to evaluate the ability of the CD40 antibody to inhibit the proliferation of B cells. Human peripheral blood PBMCs were labeled with carboxyfluorescein succinimidyl ester (CFSE), and the CD40 antibody and CD40L-mFc were added. The cells were incubated for 120 hours. The intensity of CFSE signals was used to determine the effect of the CD40 antibody on the proliferation of B cells.

PBMCs were labeled with 5 μM CFSE. After labeling, human peripheral blood PBMCs were diluted to 2×10⁵ cells/ml in complete RPMI 1640 culture medium (purchased from Shanghai Saily Biotechnology Co., Ltd.) and added into a 96-well plate at 100 μl/well. 0.5 μg/ml of CD40L-mFc was added together with 21G6 antibody (having a heavy chain sequence as set forth in SEQ ID NO: 9 and a light chain sequence as set forth in SEQ ID NO: 10), the reference antibody Iscalimab (having a heavy chain sequence as set forth in SEQ ID NO: 15 and a light chain sequence as set forth in SEQ ID NO: 16), and hIgG1LALA (purchased from Biointron), and the cells were incubated at 37 °C for 120 hours and subsequently analyzed by flow cytometry.

The results shown in FIG. 12 demonstrate that the CD40 antibody 21G6 of the present disclosure effectively inhibits CD40L-mediated proliferation of B cells, whereas the reference antibody Iscalimab exhibits a substantially weaker inhibitory effect on the proliferation of B cells than the 21G6 antibody.

It can be concluded from the above experimental results that the antibody obtained in the present disclosure is capable of binding both human and cynomolgus monkey CD40, blocking the interaction between CD40 and CD40L, inhibiting CD40 downstream activation signals, and suppressing the proliferation of B cells. Therefore, the antibody is capable of interfering with signal transduction between immune cells and regulating immune activity and inflammatory responses. These properties provide the antibody with excellent potential in treating or preventing a CD40-mediated disease and offer novel options and strategies for disease treatment and immune regulation.

The preferred embodiments of the present disclosure are described in detail above.

However, the present disclosure is not limited thereto. Without departing from the scope of the technical concept of the present disclosure, various simple modifications can be made to the technical solutions of the present disclosure, including the combination of various technical features in any other suitable manner. These simple modifications and combinations should also be regarded as the disclosed content of the present disclosure and fall within the protection scope of the present disclosure.

In the description of the present specification, the descriptions of the reference terms such as “one embodiment”, “some embodiments”, “an example”, “a specific example” and “some examples” mean that the specific features, structures, materials or characteristics described in combination with the embodiments or examples are included in at least one embodiment or example of the present disclosure. In the present specification, the schematic expressions of the above terms do not necessarily refer to the same embodiment or example. Moreover, the specific features, structures, materials or characteristics described herein may be combined in any one or more embodiments or examples in an appropriate mode. In addition, in absence of mutual contradiction, different embodiments or examples and features of the different embodiments or examples described in the present specification may be combined and integrated by those skilled in the art.

What is claimed is:

1. An antibody or antigen-binding fragment thereof, comprising:
a heavy chain variable region CDR1, a heavy chain variable region CDR2, and a heavy chain variable region CDR3 having amino acid sequences as set forth in SEQ ID NO: 1, SEQ ID NO: 2, and SEQ ID NO: 3, respectively, or amino acid sequences having at least 80% identity to SEQ ID NO: 1, SEQ ID NO: 2, and SEQ ID NO: 3, respectively; and
a light chain variable region CDR1, a light chain variable region CDR2, and a light chain variable region CDR3 having amino acid sequences as set forth in SEQ ID NO: 4, SEQ ID NO: 5, and SEQ ID NO: 6, respectively, or amino acid sequences having at least 80% identity to SEQ ID NO: 4, SEQ ID NO: 5, and SEQ ID NO: 6, respectively.

2. The antibody or antigen-binding fragment thereof according to claim 1, comprising:
a heavy chain variable region CDR1 having a sequence as set forth in SEQ ID NO: 1;
a heavy chain variable region CDR2 having a sequence as set forth in SEQ ID NO: 2;
a heavy chain variable region CDR3 having a sequence as set forth in SEQ ID NO: 3;
a light chain variable region CDR1 having a sequence as set forth in SEQ ID NO: 4;
a light chain variable region CDR2 having a sequence as set forth in SEQ ID NO: 5; and
a light chain variable region CDR3 having a sequence as set forth in SEQ ID NO: 6.

3. The antibody or antigen-binding fragment thereof according to claim 1, comprising:
a heavy chain variable region as set forth in SEQ ID NO: 7 or SEQ ID NO: 11; and/or
a light chain variable region as set forth in SEQ ID NO: 8 or SEQ ID NO: 12.

4. The antibody or antigen-binding fragment thereof according to claim 1, comprising:
1) a heavy chain variable region as set forth in SEQ ID NO: 7 and a light chain variable region as set forth in SEQ ID NO: 8; or
2) a heavy chain variable region as set forth in SEQ ID NO: 11 and a light chain variable region as set forth in SEQ ID NO: 12.

5. The antibody or antigen-binding fragment thereof according to claim 1, comprising:
a heavy chain framework region; and
a light chain framework region,
wherein at least a portion of at least one of the heavy chain framework region and the light chain framework region is from at least one of a murine antibody, a human antibody, a primate antibody, or a mutant thereof.

6. The antibody or antigen-binding fragment thereof according to claim 1, comprising:
at least one of a heavy chain constant region or a light chain constant region, wherein at least a portion of at least one of the heavy chain constant region and the light chain constant region is from at least one of a human antibody, a primate antibody, a murine antibody, or a mutant thereof.

7. The antibody or antigen-binding fragment thereof according to claim 6, wherein the light chain constant region and the heavy chain constant region are both from a murine IgG antibody or a mutant thereof, or a human IgG antibody or a mutant thereof, and
wherein the light chain constant region and the heavy chain constant region are both from a murine IgG1 antibody, a murine IgG2a antibody, a murine IgG4 antibody, or a mutant thereof, or both from a human IgG1 antibody, a human IgG2 antibody, a human IgG3 antibody, a human IgG4 antibody, or a mutant thereof.

8. The antibody or antigen-binding fragment thereof according to claim 1, comprising:
a heavy chain having an amino acid sequence as set forth in any one of SEQ ID NO: 9 and SEQ ID NO: 13; and
a light chain having an amino acid sequence as set forth in any one of SEQ ID NO: 10 and SEQ ID NO: 14.

9. The antibody or antigen-binding fragment thereof according to claim 1, comprising:
a heavy chain having an amino acid sequence as set forth in SEQ ID NO: 9; and
a light chain having an amino acid sequence as set forth in SEQ ID NO: 10, or
a heavy chain having an amino acid sequence as set forth in SEQ ID NO: 13; and
a light chain having an amino acid sequence as set forth in SEQ ID NO: 14.

10. The antibody or antigen-binding fragment thereof according to claim 1, comprising at least one of a monoclonal antibody, a polyclonal antibody, a multimeric antibody, or a CDR-grafted antibody, and
wherein the monoclonal antibody comprises at least one of a full-length antibody, an Fv antibody, a single-chain antibody, a Fab antibody, a single-domain antibody, or a minimal recognition unit.

11. The antibody or antigen-binding fragment thereof according to claim 1, being capable of binding to an amino acid sequence as set forth in SEQ ID NO: 20.

12. A nucleic acid molecule, encoding the antibody or antigen-binding fragment thereof according to claim 1.

13. An expression vector, carrying the nucleic acid molecule according to claim 12.

14. A recombinant cell, expressing the antibody or antigen-binding fragment thereof according to claim 1,
wherein the recombinant cell is a eukaryotic cell, or the recombinant cell is a mammalian cell.

15. An immunoconjugate, comprising:
the antibody or antigen-binding fragment thereof according to claim 1; and
a therapeutic agent, wherein:
the antibody or antigen-binding fragment thereof is conjugated to the therapeutic agent; and
the therapeutic agent is capable of preventing and/or treating a CD40-mediated disease, wherein the CD40-mediated disease is selected from transplant rejection, an autoimmune disease, an infectious disease, and cancer.

16. A composition, comprising the antibody or antigen-binding fragment thereof according to claim 1.

17. A medicament, comprising:
the antibody or antigen-binding fragment thereof according to claim 1; and
optionally, a pharmaceutically acceptable excipient.

18. A kit, comprising the antibody or antigen-binding fragment thereof according to claim 1.

19. A method for preventing and/or treating a CD40-mediated disease, the method comprising:
administering to a subject a pharmaceutically acceptable amount of the antibody or antigen-binding fragment thereof according to claim 1.

20. A method for detecting CD40, the method comprising:
detecting a test sample by using the antibody or antigen-binding fragment thereof according to claim 1, wherein optionally, an immune complex is formed by the antibody or antigen-binding fragment thereof and CD40 in the test sample; and
detecting whether the immune complex is present; and
determining, when detecting that the immune complex is present, that the test sample comprises CD40.
